(19) United States
(12) Patent Application Publication
(10) Pub. No.: US 2018/0016608 A1
(43) Pub. Date: Jan. 18, 2018

(54) A STRAIN FOR PRODUCTION OF GLUCOSAMINE BY MICROBIAL FERMENTATION, AND ITS METHODS

(71) Applicant: Jihong ZOU, Jiangsu (CN)

(72) Inventor: Jihong ZOU, Jiangsu (CN)

(73) Assignee: Anhui Zhengfang Biotechnology Co., Ltd., Ma'anshan City (CN)

(21) Appl. No.: 15/546,720

(22) PCT Filed: Mar. 27, 2015
(86) PCT No.: PCT/CN2015/075174

§ 371 Date: Jul. 27, 2017

(30) Foreign Application Priority Data
Jan. 27, 2015 (CN) 201510040300.7

(51) Int. Cl. C12P 19/26; C12R 1/125; C12R 1/10

(52) U.S. Cl. CPC C12P 19/26; C12R 1/10; C12R 1/125

(57) ABSTRACT

This Invention relates to, for production of N-acetyl-D-glucosamine and D-glucosamine by microbial fermentation: A nongenetic recombinant strain, preserved in the General Microbiology Center of the China Committee for Culture Collection of Microorganisms; NJ090259, a strain of Bacillus subtilis, Preservation No. CGMCC10257; and NJ091195, a strain of Bacillus licheniformis, Preservation No. CGMCC10258 and Preservation Date Dec. 29, 2014. The beneficial effects of this Invention are: Provide a new strain for production of N-acetyl-D-glucosamine and D-glucosamine, and its production methods. By this method, it may achieve stable production and supply of N-acetyl-D-glucosamine, and may also achieve non-animal-derived, safety production of N-acetyl-D-glucosamine and D-glucosamine; the method is of short production phase and low cost, and is more environmentally-friendly.

### FIELD OF TECHNOLOGY

This Invention relates the biotechnology field, specifically a strain for production of N-acetyl-D-glucosamine and D-glucosamine by microbial fermentation, and its methods.

### BACKGROUND TECHNOLOGY

N-acetyl-D-glucosamine is a monosaccharide, and is a chitin in the wall of fungla (basdiomycetes, molds, or yeasts) thallus, or a component in shell of shellfish such as crab or shrimp, or a nutritional component in a extremely low content in foods. N-acetyl-D-glucosamine has similar effects to those of glucosamine. Ingestion of a certain amount of N-acetyl-D-glucosamine may induce production of new cartilage, and inhibit episode of osteoarthritis. However in some cases, N-acetyl-D-glucosamine may also be used in treatment of osteoarthritis. Glucosamine is bitter while N-acetyl-D-glucosamine is 50% sweet of sucrose and is easily ingested. Therefore, N-acetyl-D-glucosamine has aroused an extensive concern as alternative of glucosamine.

The shall of shellfish is used as raw material for the conventional production of N-acetyl-D-glucosamine. Its production method consists of: Crush the shell of shellfish; decalcify the shell of shellfish with a dilute acid solution; remove protein with an alkali to obtain purified chitin; produce glucosamine with chitin obtained by acid hydrolysis; and then carry out acetylation of glucosamine by anhydrous acetic acid, to obtain N-acetyl-D-glucosamine The method for production of glucosamine from chitin from acid hydrolysis also include production of glucosamine with hydrochloric acid in a high concentration, using fungi dregs (such as aspergillus niger dregs for fermentation of citrate) as raw material. Refer to U.S. Pat. No. 7,049,433B2 published on May 23, 2006, which discloses glucosamine and the method for production of glucosamine by microbial fermentation.

Moreover, the conventional method also includes: (1) Production of N-acetyl-D-glucosamine with chitin produced from shrimp shell as raw material, by degradation of enzymes produced from microorganisms. Refer to U.S. Pat. No. 5,998,173 published on Dec. 7, 1999, which discloses the preparation process of N-acetyl-D-glucosamine (2) Production of N-acetyl-D-glucosamine from purification of chitin from fungi dregs (aspergillus niger dregs for citrate fermentation), by enzymolysis of enzymes produced from microorganisms (trichoderma) or by partial hydrolysis of an acid. Refer to United States Patent US20030073666A1 published on Apr. 17, 2003, which discloses N-acetyl-D-glucosamine and its preparation method. (3) Production of N-acetyl-D-glucosamine by incubation of chlorella cells infected with Chlorovirus, or of recombinant Escherichia coli originated from chlorovirus gene. Refer to United States Patent JP2004283144A published on Oct. 14, 2004, which discloses the method for preparation of glucosamine and N-acetyl-D-glucosamine (4) Production of N-acetyl-D-glucosamine by fermentation with genetically modified microorganisms, especially genetically modified Escherichia coli. Refer to United States Patent WO2004/003175 published on Jan. 8, 2004, which discloses the manufacturing process and materials of glucosamine and N-acetyl-D-glucosamine (5) N-acetyl-D-glucosamine by fermentation of trichoderma, using directly glucose as carbon source, without the need of chitin and chitin oligosaccharide as carbon source. Refer to United States Patent US20110059489A1 published on Mar. 10, 2011, which discloses the method for production of N-acetyl-D-glucosamine by microbial fermentation.

The above method for production of N-acetyl-D-glucosamine or D-glucosamine by chemical hydrolysis from the shell of shellfish or aspergillus dregs (citrate dregs) as raw material usually uses an acid or alkali solution in a high concentration, and therefore may produce a great amount of waste solution. However in extraction of D-glucosamine from the shell of shrimp or crab, the production of 1 ton of D-glucosamine may produce more than 100 tons of waste solution and a great amount of waste residues. By extraction of citrate dregs, 1 ton of D-glucosamine may be produced from 30-50 tons of citrate dregs. Moreover, the method for production of N-acetyl-D-glucosamine by degradation of chitin, derived from the shell of shellfish such as crab and shrimp is of low yield and high cost.

However, the method for production of N-acetyl-D-glucosamine by incubation of chlorella cells infected with Chlorovirus is of complicated operations, as it requires crushing cells to obtain N-acetyl-D-glucosamine The method for production of N-acetyl-D-glucosamine by genetically modified microorganisms is also of complicated operations or even impacting food safety and threatening society, as it requires appropriate measures to avoid diffusion of microorganisms in the equipment.

Additionally, the method for production of N-acetyl-D-glucosamine by trichoderma, using glucose as carbon source, had the advantages of requiring no chitin or chitin oligosaccharide as carbon source, produced from the shell of shellfish or from fungi dregs. However, as the fungi such as trichoderma are of low fermentation temperature, long time, and relatively low yield, and thus are of long production cycle, high cost, and easy contamination, seriously restricting industrial application of the method.

Therefore, aiming at the above described disadvantages in the conventional production methods for N-acetyl-D-glucosamine and D-glucosamine, it is in urgent need to find a production with industrial application potential of N-acetyl-D-glucosamine and D-glucosamine

### CONTENTS OF THE INVENTION

This Invention is intended to provide a method for production of N-acetyl-D-glucosamine and D-glucosamine by microbial fermentation, to overcome the above described disadvantages currently existing in the current technology.

This Invention is intended to achieve by the following technical schemes:

Based on one aspect of this Invention, it provide a nongenetically recombinant strain for production of N-acetyl-D-glucosamine and D-glucosamine by microbial fermentation, which is characterized by: Being preserved in the General Microbiology Center of the China Committee for Culture Collection of Microorganisms; and NJ090259, a strain of Bacillus subtilis, Preservation No. CGMCC10257 and Preservation Date Dec. 29, 2014; and NJ091195, a strain of Bacillus licheniformis, Preservation No. CGMCC10258 and Preservation Date Dec. 29, 2014.

Based on the other aspect of this Invention, the method for production of non-animal-derived N-acetyl-D-glucosamine and D-glucosamine by fermentation of the above strains, which are used as initial strain for production N-acetyl-D-glucosamine and D-glucosamine by seed cultivation and optimized medium fermentation, including the following steps:

(1) Screening, Identification, and Cultivation of Strains

Collect 50 soil samples, dilute and then smear on the plates for initial screening; using the medium for initial screening: Colloidal chitin 2.5 g/L, dipotassium hydrogen phosphate 0.7 g/L, potassium dihydrogen phosphate 0.3 g/L, magnesium sulfate 0.5 g/L, ferrous sulfate 0.01 g/L, and agar 20 g/L, pH 7.0; the culture temperature is 37° C. and the cultivation time is 72 h. Incubate to obtain individual colonies, separate colonies to obtain Bacillus subtilis and Bacillus licheniformis, cultivate it by shake flask fermentation, measure activities of chitinase in the fermented solution, and screen strains by activities of chitinase in the fermented solution.

(2) Cultivate by Fermentation

Inoculate NJ090259 (a strain of Bacillus subtilis) and NJ091195 (a strain of Bacillus licheniformis) and activated in the plate medium into the seed medium, respectively, carry out a constant-temperature shaking-bed incubation, using the solution as seed solution; inoculate the solution into the fermentation medium, carry out a constant-temperature shaking-bed incubation, centrifuge and transfer the supernatant, and measure the content of N-acetyl-D-glucosamine;

Plate medium: Colloidal chitin 30 g/L, ammonium sulfate 2 g/L, potassium dihydrogen phosphate 1.0 g/L, magnesium sulfate 0.5 g/L, sodium chloride 0.5 g/L, and agar 20 g/L, pH 6.5;

Seed medium: Peptone 5.0 g/L, beef extract 5.0 g/L, and sodium chloride 5.0 g/L, pH 7.0-7.2;

Fermentation medium: Colloidal chitin 10 g/L, glucose 10 g/L, yeast extract 3.0 g/L, MgSO₄.7H₂O 0.6 g/L, FeSO₄.7H₂O 0.01 g/L, KH₂PO₄ 0.4 g/L, K₂HPO₄ 0.6 g/L, and ZnSO₄ 0.001 g/L;

Fermentation conditions: Temperature 35° C., fermentation time 18 h, initial pH 6.5, inoculum dose 10%, and volume of loaded solution, 50 mL/250 mL;

(3) Purification of N-acetyl-D-glucosamine Fermented Solution

Transfer the supernatant obtained by centrifugation of the medium, carry an electrodialysis to remove salt, heat the salt-removed fermented solution under vacuum to concentrate to supersaturation. Cool the concentrated fermented solution, add 5 volumes of anhydrous alcohol, stir, and centrifuge to obtain a high-purity N-acetyl-glucosamine.

(4) Acidified Hydrolysis of N-acetyl-D-glucosamine

Prepare a saturated solution with N-acetyl-D-glucosamine, add 37% concentrated to a 12-16% solution, keep at 90° C. for 45-90 min, allow to cool overnight, filter to obtain crystals, wash with alcohol, vacuum-dry and measure, to obtain a high-purity D-glucosamine hydrochloride in a yield of 86%.

Further, the carbon and nitrogen sources of the said medium are provided below: The said carbon source consists of one or more of glucose, aspergillus niger dregs, trichoderma dregs, leftovers from production of black fungus, leftovers from production of glossocardia bidens, fructose, sucrose, galactose, dextrin, glycerin, starch, syrup, and molasses; the said nitrogen source consists of one or more of ammonia water, bean flour, malt, corn steep, cotton seed meal, yeast extract, ammonium chloride, ammonium sulfate, ammonium nitrate, ammonium acetate, sodium nitrate, and urea.

Further, the said fungi are one or more of Basidiomycotina, molds, and yeasts.

In this Invention, Bacillus subtilis NJ090259 and Bacillus licheniformis NJ091195 are screened from soil in different environmental conditions. The bacteriological properties of the said NJ090259 strain and NJ091195 strain are provided below:

1. Bacteriological Properties of NJ090259 Strain

Cultureological/Morphological Properties

NJ090259 strain is grown into semitransparent, pale yellow, pus-like colonies, with smooth surface; colonies round in a diameter of 4-7 mm; lowly raised, regular, radial, with lobate margin; surface creased, non-lustrous, grayish white, non-transparent, with flakes; After growth to a certain extent, it forms into creamy colonies, umbrellate, with creased surface, and large, raised in its center; single cell rod-shaped, with the two ends finely rounded, singly arranged, occasionally 2-3 cells arranged in a short chain; smeared-dried, stained cells in a size of 0.7-0.8×2-3 μm, grain positive, spores occurred by liquid fermentation for 24 h, middle or terminal, oval, inapparently swelled; uniformly opalescent in liquid matrix, without formation of bacterial membrane or ring.

Physiological and Biochemical Properties

NJ090259 strain is strictly aerobic, without any, curdling effects, being positive in catalase test, nitrate reduction test, and V-P test; being negative to phenylalanine deaminase test and yolk lecithinase test; it may decompose glucose to produce acids other than gases, and may decompose arabinose, mannitol, casein, gelatin, and starch.

2. Bacteriological Properties of NJ091195 Strain

(1) Cultureological/Morphological Properties

When incubated in a broth agar plate, NJ091195 strain forms into colonies subcircular, creamy, with dark, opaque surface, and irregular coarse margin; colonies closely attached to the medium, difficult to be picked; when incubated in liquid medium, it forms into colonies, without opalescence or precipitate; single cell short rod-like, with the two ends finely rounded, one or two cells arranged side by side, thallus 1.5-3.0 μm long and 0.6-1.0 μm wide, grain positive; spores oval, expanded in the middle, located at the center or in one side.

Physiological Properties

NJ091195 strain is positive in the catalase test, and may grown in a 7% NaCl-containing medium and at 50° C., is movable, and may use citrate and hydrolyze starch, it is negative in methyl red test, positive in gram staining, negative in lecithinase, positive in V.P test, and may use nitrate and may grown in nutritional broth at pH 5.7; it may ferment chitin, D-glucose, L-arabinose, D-xylose, and D-mannitol, and may liquefy gelatin.

The beneficial effects of this Invention are: Provide a new strain for production of N-acetyl-D-glucosamine and D-glucosamine, and its production methods. By this method, it may achieve stable production and supply of N-acetyl-D-glucosamine, and may also achieve non-animal-derived, safety production of N-acetyl-D-glucosamine and D-glucosamine; the method is of short production phase and low cost, and is more environmentally-friendly.

Actual Implementation Modes

In the following context, the technical schemes in the implementation examples of this Invention are described clearly and completely, in combination with the attached diagrams in the implementation examples of this Invention. Evidently, the described examples are just some implementation examples of this Invention, far from all the implementation examples. All other implementation examples obtained by common technicians in this field, based on the implementation examples of this Invention, fall in the scope of protection of this Invention.

Based on the implementation examples of this Invention, it provide a strain for production of N-acetyl-D-glucosamine by microbial fermentation, and its methods,

a nongenetically recombinant strain for production of N-acetyl-D-glucosamine by microbial fermentation, preserved in the General Microbiology Center of the China Committee for Culture Collection of Microorganisms; NJ090259, a strain of Bacillus subtilis, Preservation No. CGMCC10257 and Preservation Date Dec. 29, 2014; and NJ091195, a strain of Bacillus licheniformis, Preservation No. CGMCC10258 and Preservation Date Dec. 29, 2014.

### EXAMPLE 1

Based on the other aspect of this Invention, the method for production of N-acetyl-D-glucosamine and D-glucosamine by fermentation of the above strains, which are used as initial strain for production N-acetyl-D-glucosamine and D-glucosamine by seed cultivation and optimized medium fermentation, including the following steps:

(1) Screening, Identification, and Cultivation of Strains

Collect 50 soil samples, dilute by 10-3 folds and then smear on the plates for initial screening; using the medium for initial screening: Colloidal chitin 2.5 g/L, dipotassium hydrogen phosphate 0.7 g/L, potassium dihydrogen phosphate 0.3 g/L, magnesium sulfate 0.5 g/L, ferrous sulfate 0.01 g/L, and agar 20 g/L, pH 7.0; the culture temperature is 37° C. and the cultivation time is 72 h. Incubate to obtain single colonies, separate such colonies, obtain 11 strains with good growth and large & bright transparent zones of colonies. The obtained strains are identified according to the morphological characteristics of such colonies, and by grain staining and physiological & biochemical tests, to obtain 3 strains of Bacillus subtilis, and 2 strains of Bacillus licheniformis.

Activated slant strains are inoculated into a 250-mL shake flask containing 50 mL of the seed medium and incubated. The incubated liquid strains are inoculated by 10% inoculum into a 500-mL shake flask containing 100 mL of the medium and incubate. The strains are screened, according to the activities of chitinase contained in the fermented solution.

Seed medium: Peptone 10 g/L, beef extract 3 g/L, and sodium chloride 5 g/L;

Culture conditions: pH 7.4; culture temperature 37° C., shake bed rotate speed 200 rpm, and culture time 8 h;

Fermentation medium: Chitin fine powder 10 g/L, corn powder 5 g/L, starch 3 g/L, sodium nitrate 3 g/L, dipotassium hydrogen phosphate 1.05 g/L, potassium dihydrogen phosphate 0.45 g/L, sodium chloride 0.1 g/L, magnesium sulfate 0.5 g/L, and ferrous sulfate 0.03 g/L;

Culture conditions: pH 7.0; culture temperature 37° C., shake bed rotate speed 220 rpm, and culture time 72 h;

Measurement of chitinase activities in the fermented solution: Weigh 10 g of chitin fine powder, prepare with phosphate buffer to produce a 10% suspension, add a centrifuged fermented solution in the ratio of 1:1 (V/V), and incubate at 45° C. for 4 h. Then centrifuge the enzymatic hydrolysate at 3000 rpm for 10 min, transfer the supernatant, add 2 volumes of anhydrous alcohol, allow to stand overnight, and centrifuge to remove any precipitate. Concentrate the supernatant under reduced pressure to 1% reducing sugar, and measure the content of N-acetyl-D-glucosamine by HPLC.

HPLC Measurement Conditions:

Apparatuses and Equipment: Shimadzu LC-15C type High Performance Liquid Chromatograph, RID-10A Differential Refraction Detector, Chromatographic Column: Aminex Hpx-87H Column (300×7.8 mm); Mobile Phase: 5 mmol/L Sulfuric Acid Solution; flow rate: 0.6 mL/min; column temperature: 40° C.; injection volume 20 μL; measurement: RI;

Definition of enzyme activity unit: Under the enzymatic reaction conditions, the enzyme amount required by the reducing sugar equivalent to 1 μmol N-acetyl-D-glucosamine per minute is defined as one enzyme activity unit (IU).

Test Results: See Table 1 for the measured results of enzyme-producing activities from 3 strains of Bacillus subtilis, and 2 strains of Bacillus licheniformis.

TABLE 1
Chitinase Activities of 3 Strains of Bacillus subtilis
and 2 Strains of Bacillus licheniformis

| Strain and No. | Chitinase Activity (IU/mL) |
|---|---|
| No. 1 Bacillus subtilis | 0.31 |
| No. 2 Bacillus subtilis | 2.44 |
| No. 3 Bacillus subtilis | 1.34 |
| No. 1 Bacillus lincheniformis | 1.75 |
| No. 2 Bacillus lincheniformis | 0.41 |

The No. 2 Bacillus subtilis with the highest enzyme-producing activity is named as Bacillus subtilis NJ090259, and the No. 1 Bacillus Licheniformis with the highest enzyme-producing activity is named as Bacillus licheniformis NJ091195.

(2) Cultivate by Fermentation

Bacillus subtilis NJ090259, Bacillus licheniformis NJ091195, and the standard strain Bacillus licheniformis ACCC02569 are activated in the plate medium, inoculated into the seed medium, respectively, and incubated in a constant-temperature (30° C.) shake bed for 18 h, as seed solution. For inoculation, inoculate at the 1:10 ratio into the fermentation medium, incubate in a shake bed at a constant temperature of 30° C. for 72 h, centrifuge at 12000 rpm for 5 min, then transfer the supernatant, and measure the content of N-acetyl-D-glucosamine by HPLC. See Table 2 for the measured results.

Plate medium: Colloidal chitin 30 g/L, ammonium sulfate 2.0 g/L, potassium dihydrogen phosphate 1.0 g/L, magnesium sulfate 0.5 g/L, sodium chloride 0.5 g/L, and agar 20 g/L, pH 6.5;

Seed medium: Peptone 5.0 g/L, beef extract 5.0 g/L, and sodium chloride 5.0 g/L, pH 7.0-7.2;

Fermentation medium: Colloidal chitin 10 g/L, glucose 10 g/L, yeast extract 3.0 g/L, MgSO₄.7H₂O 0.6 g/L, FeSO₄.7H₂O 0.01 g/L, KH₂PO₄ 0.4 g/L, K₂HPO₄ 0.6 g/L, and ZnSO₄ 0.001 g/L;

Fermentation conditions: Temperature 35° C., fermentation time 18 h, initial pH 6.5, inoculum dose 10%, and volume of loaded solution, 50 mL/250 mL.

TABLE 2
N-acetyl-D-glucosamine Level in the Supernatant of the
Fermentation Incubation

| Strain | N-acetyl-D-glucosamine (g/L) |
|---|---|
| Bacillus licheniformi ACCC02569 | 1.13 g/L |
| Bacillus subtilis NJ090259 | 1.55 g/L |
| Bacillus licheniformis NJ091195 | 1.11 g/L |

Purification of N-acetyl-D-glucosamine Fermented Solution

Transfer the supernatant obtained by centrifugation of the medium, carry an electrodialysis to remove salt; initial salt concentration of the fermented solution in the concentrated chamber tank: 0.01 mol/L; flow rate of the fermented solution in the dilute chamber: 40 L/h; flow rate of the fermented solution in the concentrated chamber: 40 L/h. The voltage of the single membrane pair is 0.5V. The fermented solution removed of salt is heated at 50° C. under vacuum conditions (0.095 MPa) and concentrated for 8 h until oversaturation. The concentrated fermented solution is cooled to 25° C. in water at 25° C., then cooled for 1 h to 0° C. in water at 0° C. Add 5 volumes of anhydrous alcohol, and stir for 15 min. Centrifuge at 700 rpm for 15 min, add an equal volume of anhydrous alcohol and stir at 10 rpm for 0.5 h, to obtain N-acetyl-D-glucosamine crystals in a purity of 90%.

Acidified Hydrolysis of N-acetyl-D-glucosamine

Place the crude crystals of N-acetyl-D-glucosamine into a glass container, dissolve in water to prepare a saturated solution, add 37% concentrated hydrochloric acid to the final concentration of 12%, keep at 90° C. for 45 min, allow to cool to 4° C. and stand overnight, filter to obtain crystals, wash with alcohol, and vacuum-dry and measure, to obtain 97.5% D-glucosamine hydrochloride, white, in a total yield of 82%.

In another implementation example, the purification step of the N-acetyl-D-glucosamine fermented solution at Step (3) is provided below:

Transfer the supernatant obtained by centrifugation of the medium, carry an electrodialysis to remove salt; initial salt concentration of the fermented solution in the concentrated chamber tank: 0.03 mol/L; flow rate of the fermented solution in the dilute chamber: 60 L/h; flow rate of the fermented solution in the concentrated chamber: 60 L/h. The voltage of the single membrane pair is 0.5-1.4 V. The fermented solution removed of salt is heated at 65° C. under vacuum conditions (0.095 MPa) and concentrated for 11 h until oversaturation. The concentrated fermented solution is cooled to 25° C. in water at 30° C., then cooled for 2 h to 0° C. in water at 5° C. Add 5 volumes of anhydrous alcohol, and stir for 37 min. Centrifuge at 1050 rpm for 37 min, add an equal volume of anhydrous alcohol and stir at 55 rpm for 1.2 h, to obtain N-acetyl-D-glucosamine crystals in a purity of 93%.

The acid hydrolysis step of N-acetyl-D-glucosamine at Step (4) is provided below:

Place the crude crystals of N-acetyl-D-glucosamine into a glass container, dissolve in water to prepare a saturated solution, add 37% concentrated hydrochloric acid to the final concentration of 14%, keep at 90° C. for 67 min, allow to cool to 4° C. and stand overnight, filter to obtain crystals, wash with alcohol, and vacuum-dry and measure, to obtain 98.0% D-glucosamine hydrochloride, white, in a total yield of 84%.

In another implementation example, the purification step of the N-acetyl-D-glucosamine fermented solution at Step (3) is provided below:

Transfer the supernatant obtained by centrifugation of the medium, carry an electrodialysis to remove salt; initial salt concentration of the fermented solution in the concentrated chamber tank: 0.05 mol/L; flow rate of the fermented solution in the dilute chamber: 80 L/h; flow rate of the fermented solution in the concentrated chamber: 80 L/h. The voltage of the single membrane pair is 1.4V. The fermented solution removed of salt is heated at 80° C. under vacuum conditions (0.095 MPa) and concentrated for 15 h until oversaturation. The concentrated fermented solution is cooled to 25° C. in water at 35° C., then cooled for 3 h to 0° C. in water at 10° C. Add 5 volumes of anhydrous alcohol, and stir for 1 h. Centrifuge at 1500 rpm for 60 min, add an equal volume of anhydrous alcohol and stir at 100 rpm for 2 h, to obtain N-acetyl-D-glucosamine crystals in a purity of 95%.

The acid hydrolysis step of N-acetyl-D-glucosamine at Step (4) is provided below:

Place the crude crystals of N-acetyl-D-glucosamine into a glass container, dissolve in water to prepare a saturated solution, add 37% concentrated hydrochloric acid to the final concentration of 16%, keep at 90° C. for 90 min, allow to cool to 4° C. and stand overnight, filter to obtain crystals, wash with alcohol, and vacuum-dry and measure, to obtain 98.5% D-glucosamine hydrochloride, white, in a total yield of 86%.

### EXAMPLE 2

(1) Induce Mutation by Ultraviolet Radiation

Transfer 10 mL of 1×10⁷ cells/mL Bacillus subtilis NJ090259 Suspension to a 9-cm culture dish, prewarm by an ultraviolet lamp for 20 min, and place the culture dish into a magnetic stirrer, approximately 30 cm vertically from the 10-W ultraviolet lamp; start the magnetic stirrer, and irradiate for 150, 200, 250, and 300 s. After induced mutation, the bacterial suspension is allowed to stand for 1-2 h in a refrigerator, protected from light. Pick up the strain after induced mutation, and the initial strains, inoculate into the chitin medium plate, pick up the largest mutants with rapid growth rate, of which the ratio of the chitin hydrolyzation circle to the diameter of the colony is more than that of the initial strain by 10%, and measure enzyme activities.

Chitin medium: Colloidal chitin 30 g/L, ammonium sulfate 2.0 g/L, magnesium sulfate 0.5 g/L, potassium dihydrogen phosphate 1.0 g/L, and sodium chloride 0.5 g/L.

Culture conditions: pH 6.5; culture temperature 32° C. and culture time 5 days.

(2) Production of N-acetyl-D-glucosamine

By the fermentation culture method as described in the Implementation Example 1, inoculate Bacillus subtilis NJ090259 mutant modified by ultraviolet radiation, carry out a fermentation incubation test, and analyze the supernatant of the culture solution by HPLC. The results show: The culture solution obtained from the medium contains 5.5 g/L N-acetyl-D-glucosamine.

(3) Purification of N-acetyl-D-glucosamine Fermented Solution

Transfer the supernatant obtained by centrifugation of the medium, carry an electrodialysis to remove salt; initial salt concentration of the fermented solution in the concentrated chamber tank: 0.01-0.05 mol/L; flow rate of the fermented solution in the dilute chamber: 40-80 L/h; flow rate of the fermented solution in the concentrated chamber: 40-80 L/h. The voltage of the single membrane pair is 0.5-1.4 V. The fermented solution removed of salt is heated at 50-80° C. under vacuum conditions (0.095 MPa) and concentrated for 8-15 h until oversaturation. The concentrated fermented solution is cooled to 25-35° C. in water at 25° C., then cooled for 1-3 h to 0-10° C. in water at 0° C. Add 5 volumes of anhydrous alcohol, and stir for 15 min-1 h. Centrifuge at 700-1500 rpm for 15-60 min, add an equal volume of anhydrous alcohol and stir at 10-100 rpm for 0.5-2 h, to obtain N-acetyl-D-glucosamine crystals in a purity of 94%.

(4) Acidified Hydrolysis of N-acetyl-D-glucosamine

Place the crude crystals of N-acetyl-D-glucosamine into a glass container, dissolve in water to prepare a saturated solution, add (37%) concentrated hydrochloric acid to the final concentration of 12%-16%, keep at 90° C. for 45-90 min, allow to cool to 4° C. and stand overnight, filter to obtain crystals, wash with alcohol, and vacuum-dry and measure, to obtain 98.0% D-glucosamine hydrochloride, white, in a total yield of 85%.

### EXAMPLE 3

(1) Fed-Batch Fermentation of Ultraviolet-Induced Mutant of Bacillus subtilis NJ090259

The largest, ultraviolet induced mutant of Bacillus subtilis NJ090259 is activated in the plate medium, inoculated into the seed medium, and incubated in a constant-temperature (30° C.) shake bed for 18 h, as seed solution. For inoculation, inoculate in the 1:10 ratio into a 250-mL baffle conical flask containing 50 mL fermentation medium, and add 2.5 mL of the fed-batch medium at 24, 36, 48, and 60 h, respectively.

Fermentation medium: Colloidal chitin 10 g/L, glucose 10 g/L, yeast extract 3.0 g/L, MgSO₄.7H₂O 0.6 g/L, FeSO₄.7H₂O 0.01 g/L, KH₂PO₄ 0.4 g/L, K₂HPO₄ 0.6 g/L, and ZnSO₄ 0.001 g/L;

Fed-batch Medium: Colloidal chitin 100 g/L and glucose 100 g/L, pH 6.0.

Culture conditions: pH 6.5; culture temperature 35° C. and culture time 72 days. Carry out a constant-temperature shake-bed incubation.

After completion of fermentation, centrifuge at 12000 rpm for 5 min, transfer the supernatant, and measure the content of N-acetyl-D-glucosamine by HPLC.

(2) Production of N-acetyl-D-glucosamine

By the fermentation culture method as described in the Implementation Example 1, inoculate Bacillus subtilis NJ090259 mutant modified by ultraviolet radiation, carry out a fed-batch fermentation test, and analyze the supernatant of the culture solution by HPLC. The results show: The culture solution obtained from the medium contains 24.0 g/L N-acetyl-D-glucosamine.

(3) Purification of N-acetyl-D-glucosamine Fermented Solution

Transfer the supernatant obtained by centrifugation of the medium, carry an electrodialysis to remove salt; initial salt concentration of the fermented solution in the concentrated chamber tank: 0.01-0.05 mol/L; flow rate of the fermented solution in the dilute chamber: 40-80 L/h; flow rate of the fermented solution in the concentrated chamber: 40-80 L/h. The voltage of the single membrane pair is 0.5-1.4 V. The fermented solution removed of salt is heated at 50-80° C. under vacuum conditions (0.095 MPa) and concentrated for 8-15 h until oversaturation. The concentrated fermented solution is cooled to 25-35° C. in water at 25° C., then cooled for 1-3 h to 0-10° C. in water at 0° C. Add 5 volumes of anhydrous alcohol, and stir for 15 min-1 h. Centrifuge at 700-1500 rpm for 15-60 min, add an equal volume of anhydrous alcohol and stir at 10-100 rpm for 0.5-2 h, to obtain N-acetyl-D-glucosamine crystals in a purity of 96%.

(4) Acidified Hydrolysis of N-acetyl-D-glucosamine

Place the crude crystals of N-acetyl-D-glucosamine into a glass container, dissolve in water to prepare a saturated solution, add (37%) concentrated hydrochloric acid to the final concentration of 12%-16%, keep at 90° C. for 45-90 min, allow to cool to 4° C. and stand overnight, filter to obtain crystals, wash with alcohol, and vacuum-dry and measure, to obtain 98.7% D-glucosamine hydrochloride, white, in a total yield of 86.5%.

### EXAMPLE 4

(1) Induce Mutation by a Mutagenic Agent

Bacillus licheniformis NJ091195 is activated and incubate to a culture solution in the log phase. Centrifuge and transfer the supernatant, and prepare an approximately 10⁸ cells/mL bacterial suspension. Transfer 0.5 mL of 400, 600, 800, and 1000 μg/mL N-methyl-N-nitro-N-nitrosoguanidine to test tubes, and then transfer 0.5 mL each of the prepared bacterial suspension to the above test tubes. Mix well, then incubate in a water bath at 30° C. for 30 min (the treatment concentration are 200, 300, 400, and 500 μg/mL), stop the reaction by the dilution method, dilute in a dark place and smear on the chitin medium plate, and incubate at 37° C. for 5 days. Pick up the largest mutants with rapid growth rate, of which the ratio of the chitin hydrolyzation circle to the diameter of the colony is more than that of the initial strain by 10%, and measure enzyme activities.

(2) Production of N-acetyl-D-glucosamine

By the fermentation culture method as described in the Implementation Example 1, inoculate Bacillus licheniformis NJ091195 mutant induced by a mutagenic agent, carry out a fermentation incubation test, and analyze the supernatant of the culture solution by HPLC. The results show: The culture solution obtained from the medium contains 3.0 g/L N-acetyl-D-glucosamine.

(3) Purification of N-acetyl-D-glucosamine Fermented Solution

Transfer the supernatant obtained by centrifugation of the medium, carry an electrodialysis to remove salt; initial salt concentration of the fermented solution in the concentrated chamber tank: 0.01-0.05 mol/L; flow rate of the fermented solution in the dilute chamber: 40-80 L/h; flow rate of the fermented solution in the concentrated chamber: 40-80 L/h. The voltage of the single membrane pair is 0.5-1.4 V. The fermented solution removed of salt is heated at 50-80° C. under vacuum conditions (0.095 MPa) and concentrated for 8-15 h until oversaturation. The concentrated fermented solution is cooled to 25-35° C. in water at 25° C., then cooled for 1-3 h to 0-10° C. in water at 0° C. Add 5 volumes of anhydrous alcohol, and stir for 15 min-1 h. Centrifuge at 700-1500 rpm for 15-60 min, add an equal volume of anhydrous alcohol and stir at 10-100 rpm for 0.5-2 h, to obtain N-acetyl-D-glucosamine crystals in a purity of 97%.

(4) Acidified Hydrolysis of N-acetyl-D-glucosamine

Place the crude crystals of N-acetyl-D-glucosamine into a glass container, dissolve in water to prepare a saturated solution, add (37%) concentrated hydrochloric acid to the final concentration of 12%-16%, keep at 90° C. for 45-90 min, allow to cool to 4° C. and stand overnight, filter to obtain crystals, wash with alcohol, and vacuum-dry and measure, to obtain 98.6% D-glucosamine hydrochloride, white, in a total yield of 86.6%.

### EXAMPLE 5

(1) Fed-Batch Fermentation Test of Bacillus licheniformis NJ091195 Mutant Induced by a Mutagenic Agent

The largest mutant of Bacillus licheniformis NJ091195, induced by a mutagenic agent as described in Implementation Example 4, is activated in the plate medium, inoculated into the seed medium, and incubated in a constant-temperature (30° C.) shake bed for 18 h, as seed solution. For inoculation, inoculate in the 1:10 ratio into a 250-mL baffle conical flask containing 50 mL of the fermentation medium, incubate, add 2.5 mL of the fed-batch medium at 24, 36, 48, and 60 h. After completion of fermentation, centrifuge at 12000 rpm for 5 min, transfer the supernatant, and measure the content of N-acetyl-D-glucosamine by HPLC.

Fermentation medium: Colloidal chitin 10 g/L, glucose 10 g/L, yeast extract 3.0 g/L, MgSO₄.7H₂O 0.6 g/L, FeSO₄.7H₂O 0.01 g/L, KH₂PO₄ 0.4 g/L, K₂HPO₄ 0.6 g/L, and ZnSO₄ 0.001 g/L;

Fed-batch Medium: Colloidal chitin 100 g/L and glucose 100 g/L, pH 6.0.

Culture conditions: pH 6.5; culture temperature 35° C. and culture time 72 days. Carry out a constant-temperature shake-bed incubation.

(2) Production of N-acetyl-D-glucosamine

By the fermentation culture method as described in the Implementation Example 1, inoculate Bacillus licheniformis NJ091195 mutant modified by ultraviolet radiation, carry out a fed-batch fermentation test, and analyze the supernatant of the culture solution by HPLC. The results show: The culture solution obtained from the medium contains 19.0 g/L N-acetyl-D-glucosamine.

(3) Purification of N-acetyl-D-glucosamine Fermented Solution

Transfer the supernatant obtained by centrifugation of the medium, carry an electrodialysis to remove salt; initial salt concentration of the fermented solution in the concentrated chamber tank: 0.01-0.05 mol/L; flow rate of the fermented solution in the dilute chamber: 40-80 L/h; flow rate of the fermented solution in the concentrated chamber: 40-80 L/h. The voltage of the single membrane pair is 0.5-1.4 V. The fermented solution removed of salt is heated at 50-80° C. under vacuum conditions (0.095 MPa) and concentrated for 8-15 h until oversaturation. The concentrated fermented solution is cooled to 25-35° C. in water at 25° C., then cooled for 1-3 h to 0-10° C. in water at 0° C. Add 5 volumes of anhydrous alcohol, and stir for 15 min-1 h. Centrifuge at 700-1500 rpm for 15-60 min, add an equal volume of anhydrous alcohol and stir at 10-100 rpm for 0.5-2 h, to obtain N-acetyl-D-glucosamine crystals in a purity of 97.9%.

(4) Acidified Hydrolysis of N-acetyl-D-glucosamine

Place the crude crystals of N-acetyl-D-glucosamine into a glass container, dissolve in water to prepare a saturated solution, add (37%) concentrated hydrochloric acid to the final concentration of 12%-16%, keep at 90° C. for 45-90 min, allow to cool to 4° C. and stand overnight, filter to obtain crystals, wash with alcohol, and vacuum-dry and measure, to obtain 98.8% D-glucosamine hydrochloride, white, in a total yield of 87%.

HPLC Measurement Conditions of the Content of D-glucosamine Hydrochloride:

Apparatuses and Equipment: Shimadzu LC-15C type High Performance Liquid Chromatograph; Detector: Variable wavelength ultraviolet detector. Chromatographic column: NH₂ chromatographic column (4.6 mm×15 cm, 5 μm); mobile phase: Acetonitrile-phosphate buffer (60:40); flow rate: 1.5 mL/min; detection wavelength: 195 nm; column temperature: 35° C.; injection volume: 10 μL.

As the bacteriological of common bacteria are extremely variable and unstable, therefore Bacillus subtilis NJ090259 and Bacillus licheniformis NJ091195 for production of N-acetyl-D-glucosamine and D-glucosamine are mutagenized. The metagenic methods to induce mutations of bacterial strains include known spontaneous mutation method or common artificial mutation method, such as ultraviolet radiation, X-ray radiation, or mutagenic agents (such as N-methyl-N-nitro-N-nitrosoguanidine). The results show that, all strains of the above bacteria, being able to produce N-acetyl-D-glucosamine, apply to this Invention, including their spontaneous mutants and artificial mutants.

The above described examples are just good implementation examples of this Invention, and are not intended to restrict this Invention. Any modification, equivalent replacement, or improvement, made within the spirits and principles of this Invention, falls into the scope of protection of this Invention.

1. A strain for production of N-acetyl-D-glucosamine and D-glucosamine by microbial fermentation, which is characterized by: Being preserved in the General Microbiology Center of the China Committee for Culture Collection of Microorganisms; and NJ090259, a strain of Bacillus subtilis, Preservation No. CGMCC10257 and Preservation Date Dec. 29, 2014.

2. A method for production of N-acetyl-D-glucosamine by microbial fermentation, which is characterized by production of N-acetyl-D-glucosamine by seed culture and optimized medium fermentation, using NJ090259 (a strain of Bacillus subtilis) based on claim 1 as initial strain, including the following steps:
(1) Screening, Identification, and Cultivation of Strains
Collect 50 soil samples, dilute and then smear on the plates for initial screening; using the medium for initial screening: Colloidal chitin 2.5 g/L, dipotassium hydrogen phosphate 0.7 g/L, potassium dihydrogen phosphate 0.3 g/L, magnesium sulfate 0.5 g/L, ferrous sulfate 0.01 g/L, and agar 20 g/L, pH 7.0; the culture temperature is 37° C. and the cultivation time is 72 h. Incubate to obtain individual colonies, separate colonies to obtain Bacillus subtilis, cultivate it by shake flask fermentation, measure chitinase activities in the fermented solution, and screen strains by chitinase activities in the fermented solution.
(2) Cultivate by Fermentation
Inoculate NJ090259 (a strain of Bacillus subtilis) activated in the plate medium into the seed medium, carry out a constant-temperature shaking-bed incubation, using the solution as seed solution; inoculate the solution into the fermentation medium, carry out a constant-temperature shaking-bed incubation, centrifuge and transfer the supernatant, and measure the content of N-acetyl-D-glucosamine;
Plate medium: Colloidal chitin 30 g/L, ammonium sulfate 2 g/L, potassium dihydrogen phosphate 1.0 g/L, magnesium sulfate 0.5 g/L, sodium chloride 0.5 g/L, and agar 20 g/L, pH 6.5;
Seed medium: Peptone 5.0 g/L, beef extract 5.0 g/L, and sodium chloride 5.0 g/L, pH 7.0-7.2;
Fermentation medium: Colloidal chitin 10 g/L, glucose 10 g/L, yeast extract 3.0 g/L, MgSO₄.7H₂O 0.6 g/L, FeSO₄.7H₂O 0.01 g/L, KH₂PO₄ 0.4 g/L, K₂HPO₄ 0.6 g/L, and ZnSO₄ 0.001 g/L;
Fermentation conditions: Temperature 35° C., fermentation time 18 h, initial pH 6.5, inoculum dose 10%, and volume of loaded solution, 50 mL/250 mL;
(3) Purification of N-acetyl-D-glucosamine Fermented Solution
Transfer the supernatant obtained by centrifugation of the medium, carry an electrodialysis to remove salt, heat the salt-removed fermented solution under vacuum to concentrate to supersaturation. Cool the concentrated fermented solution, add 5 volumes of anhydrous alcohol, stir, and centrifuge to obtain a high-purity N-acetyl-glucosamine.

3. A method for production of D-glucosamine by microbial fermentation, which is characterized by production of D-glucosamine by seed culture and optimized medium fermentation, using NJ090259 (a strain of Bacillus subtilis) based on claim 1, including the following steps:
(1) Screening, Identification, and Cultivation of Strains
Collect 50 soil samples, dilute and then smear on the plates for initial screening; using the medium for initial screening: Colloidal chitin 2.5 g/L, dipotassium hydrogen phosphate 0.7 g/L, potassium dihydrogen phosphate 0.3 g/L, magnesium sulfate 0.5 g/L, ferrous sulfate 0.01 g/L, and agar 20 g/L, pH 7.0; the culture temperature is 37° C. and the cultivation time is 72 h. Incubate to obtain individual colonies, separate colonies to obtain Bacillus subtilis, cultivate it by shake flask fermentation, measure chitinase activities in the fermented solution, and screen strains by chitinase activities in the fermented solution.
(2) Cultivate by Fermentation
Inoculate NJ090259 (a strain of Bacillus subtilis) activated in the plate medium into the seed medium, carry out a constant-temperature shaking-bed incubation, using the solution as seed solution; inoculate the solution into the fermentation medium, carry out a constant-temperature shaking-bed incubation, centrifuge and transfer the supernatant, and measure the content of N-acetyl-D-glucosamine;
Plate medium: Colloidal chitin 30 g/L, ammonium sulfate 2 g/L, potassium dihydrogen phosphate 1.0 g/L, magnesium sulfate 0.5 g/L, sodium chloride 0.5 g/L, and agar 20 g/L, pH 6.5;
Seed medium: Peptone 5.0 g/L, beef extract 5.0 g/L, and sodium chloride 5.0 g/L, pH 7.0-7.2;
Fermentation medium: Colloidal chitin 10 g/L, glucose 10 g/L, yeast extract 3.0 g/L, MgSO₄.7H₂O 0.6 g/L, FeSO₄.7H₂O 0.01 g/L, KH₂PO₄ 0.4 g/L, K₂HPO₄ 0.6 g/L, and ZnSO₄ 0.001 g/L;
Fermentation conditions: Temperature 35° C., fermentation time 18 h, initial pH 6.5, inoculum dose 10%, and volume of loaded solution, 50 mL/250 mL;
(3) Purification of N-acetyl-D-glucosamine Fermented Solution
Transfer the supernatant obtained by centrifugation of the medium, carry an electrodialysis to remove salt, heat the salt-removed fermented solution under vacuum to concentrate to supersaturation. Cool the concentrated fermented solution, add 5 volumes of anhydrous alcohol, stir, and centrifuge to obtain a high-purity N-acetyl-glucosamine.
(4) Acidified Hydrolysis of N-acetyl-D-glucosamine
Prepare a saturated solution with N-acetyl-D-glucosamine, add 37% concentrated hydrochloric acid to a final concentration of 12-16%, keep at 90° C. for 45-90 min, allow to cool overnight, filter to obtain crystals, wash with alcohol, vacuum-dry and measure, to obtain a high-purity D-glucosamine hydrochloride.

4. A strain for production of N-acetyl-D-glucosamine and D-glucosamine by microbial fermentation, which is characterized by: Being preserved in the General Microbiology Center of the China Committee for Culture Collection of Microorganisms (CCCCM); and NJ091195, a strain of Bacillus licheniformis, Preservation No. CGMCC10258 and Preservation Date Dec. 29, 2014.

5. A method for production of N-acetyl-D-glucosamine by microbial fermentation, which is characterized by production of N-acetyl-D-glucosamine by seed culture and optimized medium fermentation, using NJ091195 (a strain of Bacillus subtilis) based on claim 4 as initial strain, including the following steps:
(1) Screening, Identification, and Cultivation of Strains
Collect 50 soil samples, dilute and then smear on the plates for initial screening; using the medium for initial screening: Colloidal chitin 2.5 g/L, dipotassium hydrogen phosphate 0.7 g/L, potassium dihydrogen phosphate 0.3 g/L, magnesium sulfate 0.5 g/L, ferrous sulfate 0.01 g/L, and agar 20 g/L, pH 7.0; the culture temperature is 37° C. and the cultivation time is 72 h. Incubate to obtain individual colonies, separate colonies to obtain Bacillus licheniformis, cultivate it by shake flask fermentation, measure chitinase activities in the fermented solution, and screen strains by activities of chitinase in the fermented solution.
(2) Cultivate by Fermentation
Inoculate NJ091195 (a strain of Bacillus licheniformis) activated in the plate medium into the seed medium, carry out a constant-temperature shaking-bed incubation, using the solution as seed solution; inoculate the solution into the fermentation medium, carry out a constant-temperature shaking-bed incubation, centrifuge and transfer the supernatant, and measure the content of N-acetyl-D-glucosamine;
Plate medium: Colloidal chitin 30 g/L, ammonium sulfate 2 g/L, potassium dihydrogen phosphate 1.0 g/L, magnesium sulfate 0.5 g/L, sodium chloride 0.5 g/L, and agar 20 g/L, pH 6.5;
Seed medium: Peptone 5.0 g/L, beef extract 5.0 g/L, and sodium chloride 5.0 g/L, pH 7.0-7.2;
Fermentation medium: Colloidal chitin 10 g/L, glucose 10 g/L, yeast extract 3.0 g/L, MgSO₄.7H₂O 0.6 g/L, FeSO₄.7H₂O 0.01 g/L, KH₂PO₄ 0.4 g/L, K₂HPO₄ 0.6 g/L, and ZnSO₄ 0.001 g/L;
Fermentation conditions: Temperature 35° C., fermentation time 18 h, initial pH 6.5, inoculum dose 10%, and volume of loaded solution, 50 mL/250 mL;
(3) Purification of N-acetyl-D-glucosamine Fermented Solution
Transfer the supernatant obtained by centrifugation of the medium, carry an electrodialysis to remove salt, heat the salt-removed fermented solution under vacuum to concentrate to supersaturation. Cool the concentrated fermented solution, add 5 volumes of anhydrous alcohol, stir, and centrifuge to obtain a high-purity N-acetyl-glucosamine.

6. A method for production of D-glucosamine by microbial fermentation, which is characterized by production of N-acetyl-D-glucosamine by seed culture and optimized medium fermentation, using NJ091195 (a strain of Bacillus subtilis) based on claim 4 as initial strain, including the following steps:
(1) Screening, Identification, and Cultivation of Strains
Collect 50 soil samples, dilute and then smear on the plates for initial screening; using the medium for initial screening: Colloidal chitin 2.5 g/L, dipotassium hydrogen phosphate 0.7 g/L, potassium dihydrogen phosphate 0.3 g/L, magnesium sulfate 0.5 g/L, ferrous sulfate 0.01 g/L, and agar 20 g/L, pH 7.0; the culture temperature is 37° C. and the cultivation time is 72 h. Incubate to obtain individual colonies, separate colonies to obtain Bacillus licheniformis, cultivate it by shake flask fermentation, measure chitinase activities in the fermented solution, and screen strains by activities of chitinase in the fermented solution.
(2) Cultivate by Fermentation
Inoculate NJ091195 (a strain of Bacillus licheniformis) activated in the plate medium into the seed medium, carry out a constant-temperature shaking-bed incubation, using the solution as seed solution; inoculate the solution into the fermentation medium, carry out a constant-temperature shaking-bed incubation, centrifuge and transfer the supernatant, and measure the content of N-acetyl-D-glucosamine;
Plate medium: Colloidal chitin 30 g/L, ammonium sulfate 2 g/L, potassium dihydrogen phosphate 1.0 g/L, magnesium sulfate 0.5 g/L, sodium chloride 0.5 g/L, and agar 20 g/L, pH 6.5;
Seed medium: Peptone 5.0 g/L, beef extract 5.0 g/L, and sodium chloride 5.0 g/L, pH 7.0-7.2;
Fermentation medium: Colloidal chitin 10 g/L, glucose 10 g/L, yeast extract 3.0 g/L, MgSO₄.7H₂O 0.6 g/L, FeSO₄.7H₂O 0.01 g/L, KH₂PO₄ 0.4 g/L, K₂HPO₄ 0.6 g/L, and ZnSO₄ 0.001 g/L;
Fermentation conditions: Temperature 35° C., fermentation time 18 h, initial pH 6.5, inoculum dose 10%, and volume of loaded solution, 50 mL/250 mL;
(3) Purification of N-acetyl-D-glucosamine Fermented Solution
Transfer the supernatant obtained by centrifugation of the medium, carry an electrodialysis to remove salt, heat the salt-removed fermented solution under vacuum to concentrate to supersaturation. Cool the concentrated fermented solution, add 5 volumes of anhydrous alcohol, stir, and centrifuge to obtain a high-purity N-acetyl-glucosamine.
(4) Acidified Hydrolysis of N-acetyl-D-glucosamine
Prepare a saturated solution with N-acetyl-D-glucosamine, add 37% concentrated hydrochloric acid to a final concentration of 12-16%, keep at 90° C. for 45-90 min, allow to cool overnight, filter to obtain crystals, wash with alcohol, vacuum-dry and measure, to obtain a high-purity D-glucosamine hydrochloride.

7. A method for production of N-acetyl-D-glucosamine by microbial fermentation, based on claim 2, which is characterized by that the carbon and nitrogen sources of the said medium provided below: The said carbon source consists of one or more of glucose, aspergillus niger dregs, trichoderma dregs, leftovers from production of black fungus, leftovers from production of glossocardia bidens, fructose, sucrose, galactose, dextrin, glycerin, starch, syrup, and molasses; the said nitrogen source consists of one or more of ammonia water, bean flour, malt, corn steep, cotton seed meal, yeast extract, ammonium chloride, ammonium sulfate, ammonium nitrate, ammonium acetate, sodium nitrate, and urea.

8. A method for production of D-glucosamine by microbial fermentation, based on claim 3, which is characterized by that the carbon and nitrogen sources of the said medium provided below: The said carbon source consists of one or more of glucose, aspergillus niger dregs, trichoderma dregs, leftovers from production of black fungus, leftovers from production of glossocardia bidens, fructose, sucrose, galactose, dextrin, glycerin, starch, syrup, and molasses; the said nitrogen source consists of one or more of ammonia water, bean flour, malt, corn steep, cotton seed meal, yeast extract, ammonium chloride, ammonium sulfate, ammonium nitrate, ammonium acetate, sodium nitrate, and urea.

9. A method for production of N-acetyl-D-glucosamine by microbial fermentation, based on claim 5, which is characterized by that the carbon and nitrogen sources of the said medium provided below: The said carbon source consists of one or more of glucose, aspergillus niger dregs, trichoderma dregs, leftovers from production of black fungus, leftovers from production of glossocardia bidens, fructose, sucrose, galactose, dextrin, glycerin, starch, syrup, and molasses; the said nitrogen source consists of one or more of ammonia water, bean flour, malt, corn steep, cotton seed meal, yeast extract, ammonium chloride, ammonium sulfate, ammonium nitrate, ammonium acetate, sodium nitrate, and urea.

10. A method for production of D-glucosamine by microbial fermentation, based on claim 6, which is characterized by that the carbon and nitrogen sources of the said medium provided below: The said carbon source consists of one or more of glucose, aspergillus niger dregs, trichoderma dregs, leftovers from production of black fungus, leftovers from production of glossocardia bidens, fructose, sucrose, galactose, dextrin, glycerin, starch, syrup, and molasses; the said nitrogen source consists of one or more of ammonia water, bean flour, malt, corn steep, cotton seed meal, yeast extract, ammonium chloride, ammonium sulfate, ammonium nitrate, ammonium acetate, sodium nitrate, and urea.
